(19) United States
(12) Patent Application Publication
(10) Pub. No.: US 2005/0228394 A1
(43) Pub. Date: Oct. 13, 2005

(54) Suction inserter

(76) Inventors: Diane Bihary, Fort Wayne, IN (US); Anthony Charles Siebeneck, Ottawa, OH (US); Wayne M. Goldstein, Highland Park, IL (US)

Correspondence Address: MAGINOT, MOORE & BECK;BANK ONE CENTER/TOWER, 1111 MONUMENT CIRCLE, INDIANAPOLIS, IN, 46204, US

(21) Appl. No.: 10/814,557

(22) Filed: Mar. 31, 2004

(51) Int. Cl. A61B 17/58

(52) U.S. Cl. 606/91

(57) ABSTRACT

A hand held instrument for inserting an acetabular liner into acetabular cup is provided. The instrument includes a bulb syringe, a shaft and a head configured to sealingly engage an acetabular liner to create a sealed area between the acetabular liner and the head. The bulb syringe is used to create a vacuum in the sealed area and/or to force air into the sealed area. After the acetabular liner is placed into an acetabular cup, the vacuum is released and/or air is forced into the sealed area to force the acetabular liner off of the head.

### FIELD OF THE INVENTION

The present invention relates to surgical instruments, and particularly to an instrument for positioning an acetabular insert.

### BACKGROUND OF THE INVENTION

Joint arthroplasty is a well known surgical procedure by which a diseased and/or damaged natural joint is replaced by a prosthetic joint. Joint arthroplasty is commonly performed for hips, knees, elbows, and other joints. The health and condition of the joint to be replaced dictate the type(s) of prosthesis necessary to replace the natural joint. Prosthetic components that are used in a hip arthroplasty include acetabular cups and acetabular liners. FIG. 1 shows acetabular cup 10 and acetabular liner 12. An acetabular cup is implanted in the acetabular cavity in the pelvis to replace the natural acetabulum. The acetabulum receives the head or ball of a femoral component. FIG. 2 depicts the left hip of an individual, showing the general location of the pelvis 14 and femur 16. Replacement of the acetabulum is necessary when there is an inadequate articulation surface for a head or ball of a femoral component.

To implant an acetabular cup, a cavity is reamed in the pelvis. The reamed cavity generally conforms to an outer surface of the acetabular cup. The acetabular cup is then inserted into the formed cavity and secured. The acetabular cup is positioned in the pelvis at a fixed orientation in the acetabulum so as to emulate the patient's natural anatomy. The implanted cup should remain stable to prevent erosion of the surrounding bone and to inhibit generation of excessive wear debris in the prosthetic joint. Next, a liner is inserted into the acetabular cup. The liner acts as a bearing surface against which the ball or head of the femoral component presses. FIG. 3 shows acetabular cup 10 and acetabular liner 12 inserted into pelvis 14.

Various methods and techniques have been used to secure an acetabular cup within a formed acetabular cavity. One such method includes the use of bone cement to secure the acetabular cup to the acetabulum. Another technique utilizes an acetabular cup having holes for receiving screws, or other types of fasteners, to affix the acetabular cup to bone. A further method includes the implantation of an acetabular cup having an outer surface with various surface features to enhance fixation of the cup within the acetabular cavity. Two or more of these methods may be used in conjunction with each other to secure the acetabular cup to the acetabulum.

No matter which technique(s) are used to secure the acetabular cup, proper initial positioning of the acetabular cup within the reamed cavity is critical to the proper functioning of the prosthetic component. The positioning of the cup is complicated by the fact that incisions used in hip or femoral operations may be quite deep, with the implantation site remote from the initial incision location. Moreover, it is desired to keep the incision as small as possible both for improved healing as well as aesthetic purposes. An exemplary incision and various surgical instruments that may be used during implantation of an acetabular cup are shown in FIG. 4. As shown in FIG. 4, a surgeon's view of the implantation site may be relatively obstructed.

The implantation of a liner presents many of the same difficulties as the implantation of the cup. The instruments used to keep the incision site open crowd the incision area and obscure the surgeon's vision. Thus, the liner must be manipulated into position in a very confined space. Moreover, care must be taken to ensure that no tissue is trapped between the acetabular cup and the acetabular liner as the liner is being inserted. Obviously, obscuring the vision of the surgeon hinders the procedure and can result in undesired delays.

Accordingly, a number of instruments have been designed to assist in the proper placement of the acetabular implants. One such instrument uses tongs to grasp the outer rim of an acetabular liner. Typically, an acetabular liner to be placed with such an instrument must be provided with grooves about the outer rim of the acetabular liner to provide for proper engagement of the acetabular liner with the instrument. Thus, specially produced acetabular liners are required. Moreover, because the instrument must extend beyond the rim of the acetabular liner in order to properly engage the acetabular cup, a surgeon's view of the implantation site may be negatively impacted. Additionally, the release mechanism is typically located such that a surgeon's hand must be placed over the incision site, further obscuring the surgeon's vision.

Another instrument that has been used in placing acetabular cups is a suction based device with a head that sealingly fits to the rim of the acetabular liner. Aspirant suction that is typically available in surgical rooms is provided to the device through a tube and a vacuum is drawn between the acetabular cup and the head of the instrument. This instrument advantageously provides for secure engagement of the acetabular cup by the instrument. This instrument also provides a significant advantage over approaches which rely on physically engaging the acetabular cup, such as by threaded engagement, in that separation is easily accomplished by releasing the vacuum. However, some portion of the instrument must still extend over the rim of the acetabular cup in order to provide a sealing surface. Additionally, the connection of suction tubing to the device hinders the maneuverability of the device. Moreover, the tubing creates an obstacle for medical personnel that may hinder movement in and around the device.

It may be possible to modify an aspirant suction instrument for use with an acetabular liner. However, acetabular liners are significantly lighter than acetabular cups. Thus, placement of acetabular liners requires much lower levels of vacuum than the vacuum associated with the above vacuum based device. Accordingly, one instrument used for inserting acetabular liners that mitigates some of the limitations of the aspirant based suction devices is an instrument that uses a suction cup at the end of a wand.

A suction cup works merely by pressing a resilient cup shaped surface against the inner portion of an acetabular liner, thereby expelling air from the suction cup and creating a vacuum between the suction cup and the acetabular liner. Such a device is very inexpensive while providing increased maneuverability and decreased bulk since no tubing must be connected to the device. Because there are no tubes connected, surgical room clutter is not exacerbated. Unfortunately, such a device has limited usefulness. Many acetabular liners are now made of ceramic and other heavy materials, and the limited vacuum available with the suction cup device is insufficient to provide a secure grasp of the acetabular liner. Moreover, once the acetabular liner is installed, there is no easy way to release the suction cup. Rather, the wand must be manipulated until the seal with the acetabular liner is broken. In attempting to release the liner, the acetabular liner may be knocked out of its proper position.

What is needed therefore is an apparatus and method for positioning an acetabular liner which overcomes one or more of the above-mentioned disadvantages.

### SUMMARY OF THE INVENTION

In one embodiment of the invention, a suction inserter comprises a shaft with an internal channel sealing connected to a bulb syringe at one end portion and a head at the second end portion. The head is configured to sealingly fit within an acetabular liner. The shaft is bent between the first end portion and the second end portion to allow improved viewing when inserting an acetabular liner.

In another embodiment, the head of a suction inserter includes an internal channel providing communication between the internal channel of a shaft and the end portion of a head. The head includes two o-rings to provide sealing engagement with an acetabular liner and to provide a stable base for the liner when a vacuum is drawn between the acetabular liner and the head. A secondary internal channel is provided between the two o-rings to ensure the acetabular liner is firmly seated on both o-rings.

In certain embodiments, various valves may be provided separately or in combination. The valves include isolation valves, vent valves and stop check valves. The use of various valves in various combinations allows for a number of alternative methods of using the disclosed suction inverter.

In one embodiment, a handheld instrument for insertion of an acetabular liner into an acetabular cup comprises (i) a shaft having an internal channel therethrough and a first and a second end portion, the first end portion is configured to sealingly engage with a bulb syringe and (ii) a head having a curvilinear outer perimeter and operably attached to the second end portion of the shaft and having an inner channel therethrough operably connected to the internal channel of the shaft, the curvilinear outer perimeter of the head sized to at least partially fit within an acetabular liner.

One embodiment of the present invention relates to a method of inserting an acetabular liner into an acetabular cup by performing the steps of positioning an acetabular liner on the head of an instrument, placing the acetabular liner into an acetabular cup, and squeezing for a first time a bulb syringe connected to the instrument to expel air out of the bulb syringe.

Another embodiment of the present invention relates to a method of inserting an acetabular liner into an acetabular cup by performing the steps of squeezing the bulb syringe of an instrument a first time, sealingly engaging the head of the instrument within an acetabular liner so as to create a sealed area between the head and the acetabular liner, drawing a vacuum in the sealed area, placing the acetabular liner in an acetabular cup, removing the vacuum from the sealed area, and separating the head from the acetabular liner in the acetabular cup.

It is one object of the present invention to provide an inexpensive and easily manufactured instrument for insertion of an acetabular liner. A further object is to provide an insertion instrument that securely holds the acetabular liner without unduly impeding a surgeon's view of the insertion location. Yet another object is to allow for easily disengaging the insertion instrument once an acetabular liner is placed within an acetabular cup.

These objects and certain benefits of the invention can be ascertained from the following written description taken together with the accompanying figures.

### DESCRIPTION OF THE FIGURES

FIG. 1 is a perspective view of an exemplary acetabular cup and an acetabular liner of the prior art.

FIG. 2 is an elevational partially cutaway view of the hip of an individual showing a pelvis and a femur.

FIG. 3 is a perspective view of the acetabular cup and acetabular liner of FIG. 1 implanted in the pelvis of FIG. 2.

FIG. 4 is a perspective view of a surgical incision during a hip replacement operation.

FIG. 5 is an elevational view of a suction inserter that incorporates features of the present invention.

FIG. 6 is an elevational view of the head of the suction inserter of FIG. 5.

FIG. 7 is a cross sectional view of the head taken along line A-A of FIG. 6.

FIG. 8 is a cross sectional view of an acetabular liner frictionally engaged with the head of FIG. 5, also showing a portion of the shaft of the suction inserter of FIG. 5.

FIG. 9 is an elevational view of a female luer used to provide an airtight seal between the bulb syringe and the shaft of the suction inserter of FIG. 5.

FIG. 10 is a view similar to FIG. 8, except showing that the acetabular liner has been drawn toward the head of the suction inserter by a vacuum.

FIG. 11 is an alternative embodiment of a suction inserter that incorporates features of the present invention.

FIG. 12 is an alternative hand held vacuum producing device that may be used with a suction inserter incorporating features of the present invention.

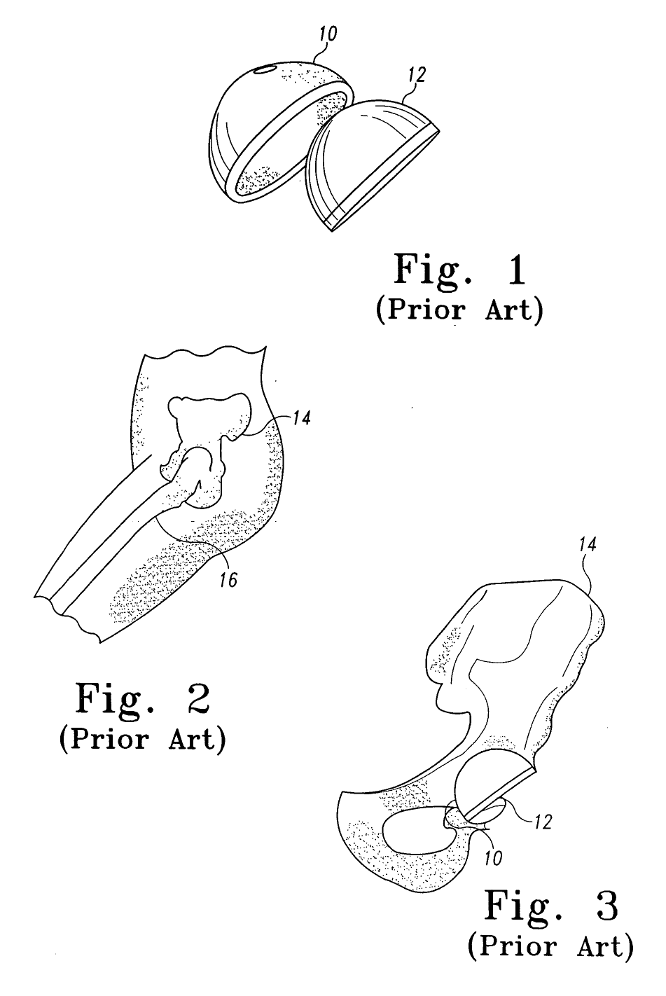

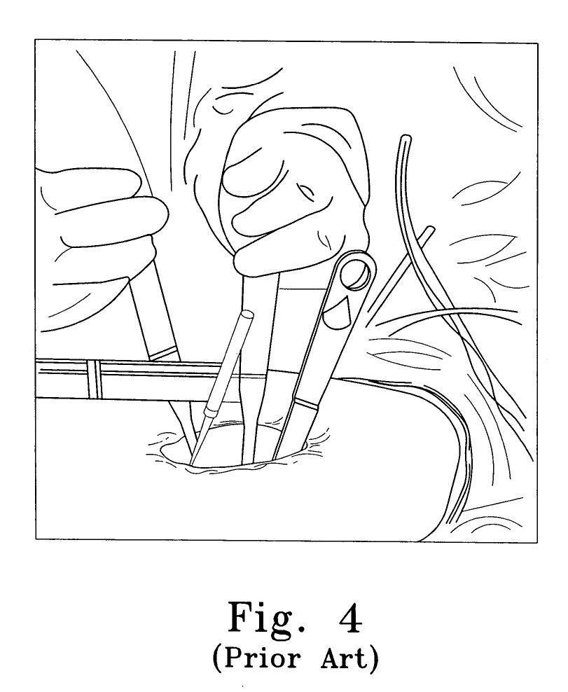

### DESCRIPTION OF THE PREFERRED EMBODIMENTS

For the purposes of promoting an understanding of the principles of the invention, reference will now be made to the embodiments illustrated in the drawings and described in the following written description. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

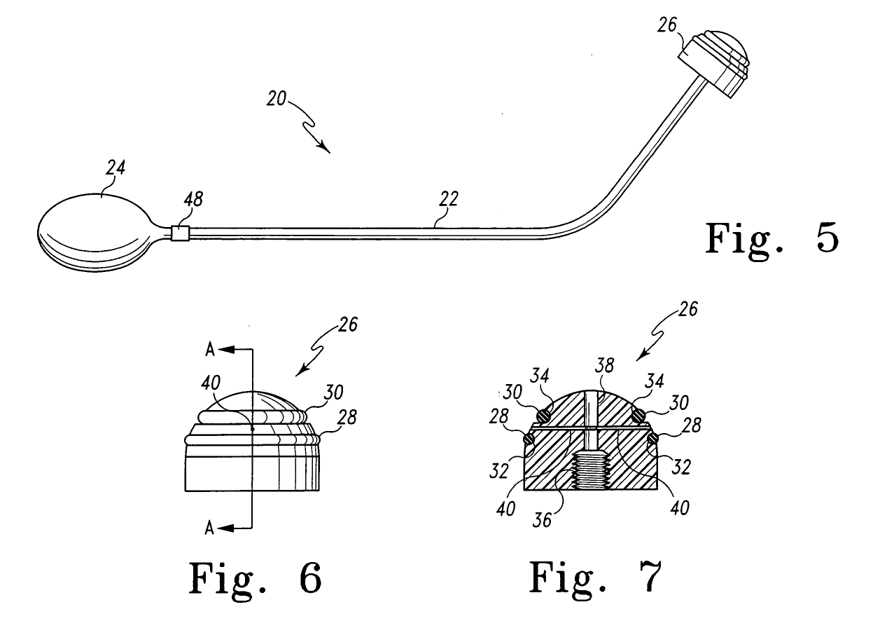

Referring generally to FIG. 5, a suction inserter for insertion of an acetabular liner into an acetabular cup is shown, hereinafter generally designated by reference numeral 20. Suction inserter 20 comprises bent shaft 22, bulb syringe 24, and head 26. Head 26 comprises o-ring 28 and o-ring 30 as shown in FIG. 6. FIG. 7, which is cross sectional view of head 26 taken along line A-A of FIG. 6, shows o-rings 28 and 30 located within grooves 32 and 34 respectively. Head 26 further comprises threaded chamber 36, inner channel 38 and secondary inner channel 40.

Head 26 in this embodiment is constructed from a medical grade acetal such as is commercially available under the trade name DELRIN® or CELCON®). Forming of head 26 is accomplished by first selecting a desired diameter of stock acetal. In this embodiment, the desired diameter of stock corresponds to the diameter of the acetabular liner to be inserted. Acetabular liners are typically made with diameters of 26 mm, 28 mm, 32 mm, 36 mm or 38 mm. Thus, in accordance with one embodiment of the invention, a kit includes a bulb syringe, a shaft, and at least two heads of different diameters, each of the heads configured to fit within a liner having a diameter of 26 mm, 28 mm, 32 mm, 36 mm or 38 mm. The kit may include other sizes either alternatively or in addition to the just mentioned sizes and are within the scope of the present invention.

Once the desired stock is selected, threaded chamber 36, inner channel 38 and secondary inner channel 40 are formed in accordance with methods well known in the relevant art. The portion of head 26 that is to be placed within an acetabular liner is then reduced such that the acetal portion of the head is spaced apart from the acetabular liner when head 26 is sealingly inserted into the acetabular liner. Next, grooves for o-rings 28 and 30 are formed. The depth of each groove is selected in conjunction with the size of the o-ring to be used in that groove. In one embodiment, the grooves and o-rings are sized such that insertion of head 26 within an acetabular liner results in a light frictional engagement of the o-rings with the acetabular liner, providing an airtight seal.

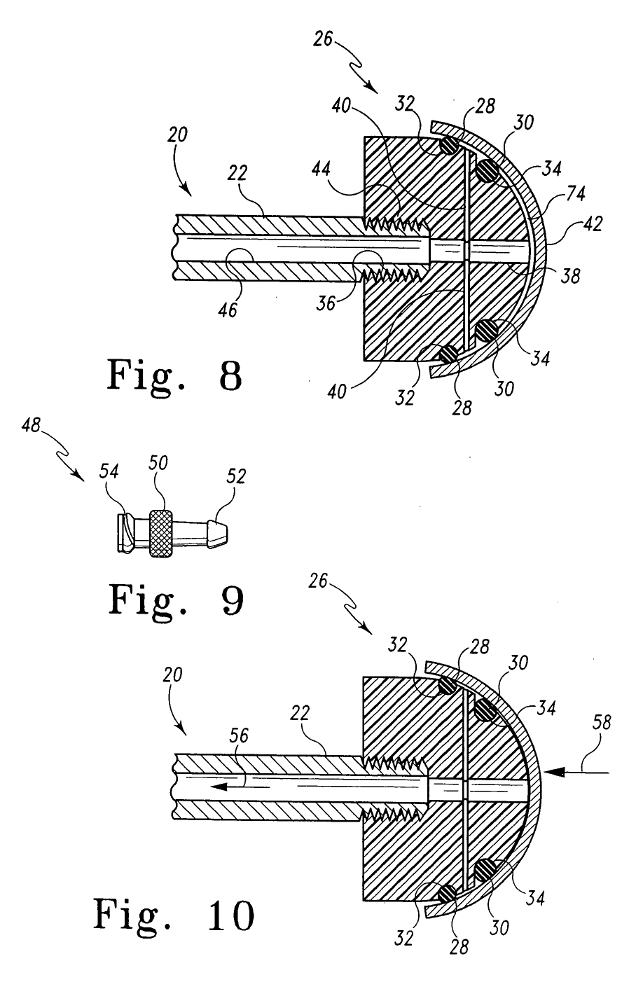

Use of a second o-ring provides increased stability between suction inserter 20 and a held acetabular liner. With reference to FIG. 8, which shows acetabular liner frictionally engaged with head 26, both o-ring 28 and o-ring 30 contact acetabular liner 42. The two contact areas effectively provide a larger base upon which the acetabular liner is seated. Thus, the acetabular liner is more securely held by head 26.

Continuing with FIG. 8, shaft 22 is threaded at first end portion 44, and defines internal channel 46. Shaft 22 in this embodiment is constructed of 316 stainless steel, having an outside diameter of about ¼ inch and is 12.25 inches long. Internal channel 46 has a diameter of between about 1/16 inch and about 3/32 inch. The bend in shaft 22 is obtained by mechanically bending shaft 22 to the desired angle. In a preferred embodiment, shaft 22 is bent between about 20 degrees and about 45 degrees. In this embodiment, shaft 22 is bent about 30 degrees. First end portion 44 is threaded so as to engage the threads of threaded chamber 36. Thus, when head 26 is screwed onto shaft 22, first end portion 44 sealingly engages threaded chamber 36.

Bulb syringe 24 which in this embodiment is a medical grade 2 ounce bulb syringe made from heavy duty rubber, is sealingly engaged to shaft 22 by female luer 48, shown in FIG. 9. Luer 48 is made from 316 stainless steel and serves as an adapter from shaft 22 to bulb syringe 24. Luer 48 comprises knob 50, male portion 52 and threaded portion 54. Such luers are commercially available under the trade name POPPER®. Male portion 52 may be modified as necessary to fit within internal channel 46 of shaft 22. Male portion 52 is then inserted into shaft 22 and knob 50 is welded to shaft 22 to provide an airtight seal. Threaded portion 54 comprises a special thread that is commonly available in medical grade bulb syringes. Thus, sealing engagement between bulb syringe 24 and luer 48 is accomplished by screwing bulb syringe 24 onto luer 48.

The operation of suction inserter 20 is more fully provided below, however, a brief summary of the use of suction inserter 20 is provided at this point to assist in understanding of the instrument. With reference to FIG. 5, bulb syringe 24 is used to create a vacuum. This is accomplished by gripping and squeezing bulb syringe 24 in a hand so as to expel air from bulb syringe 24 through internal channel 46 and inner channel 38 of head 26, and out of suction inserter 20. Acetabular liner 42 is then placed in sealing engagement onto head 26 as shown in FIG. 8. The squeezing grip on bulb syringe 24 is then released, and the resilient nature of bulb syringe 24 creates a vacuum that is transferred through internal channel 46 and inner channel 38 to the inner surface of acetabular liner 42. The vacuum is also transferred to the area between acetabular liner 42 and head 26 by o-ring 28 and o-ring 30 through secondary inner channel 40. Thus, acetabular liner 42 is pulled closer to head 26 from its position shown in FIG. 8 to its position in FIG. 10 by the vacuum caused by bulb syringe 24 as indicated by arrows 56 and 58 in FIG. 10. Acetabular liner 42 is thus held firmly by suction inserter 20 both by friction and by vacuum.

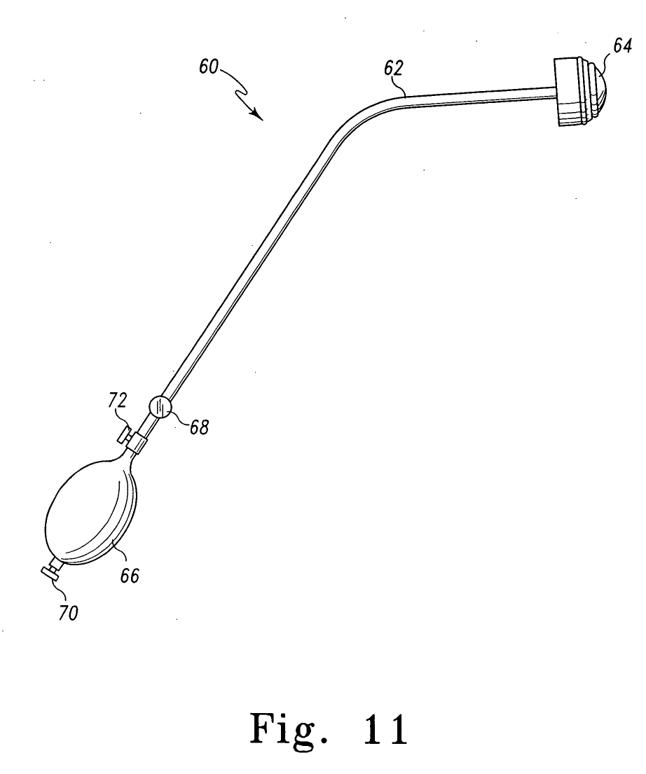

An alternative embodiment of a suction instrument in accordance with the present invention is shown in FIG. 11. In this embodiment, suction inserter 60 comprises shaft 62 which is sealingly engaged with head 64 and bulb syringe 66. Suction inserter 60 includes isolation valve 68. Bulb syringe 66 includes vent valve 70 and stop check valve 72.

Isolation valve 68 has an open and a shut position. When isolation valve 68 is open, air is allowed to pass through isolation valve 68 between bulb syringe 66 and an internal channel (not shown) defined in shaft 62. When isolation valve 68 is shut, air is not allowed to pass through isolation valve 68.

Vent valve 70 has an open and a shut position. When vent valve 70 is open, air is allowed to pass through vent valve 70 between bulb syringe 66 and the atmosphere. When vent valve 70 is shut, air is not allowed to pass through vent valve 70.

Stop check valve 72 has a released and a stop position. When stop check valve 72 is in the released position, air is allowed to pass through stop check valve 72 from bulb syringe 66 to the atmosphere; however, air is not allowed to pass from atmosphere through stop check valve 72 into bulb syringe 66. When stop check valve 72 is in the stop position, air is not allowed to pass through stop check valve 72 in either direction.

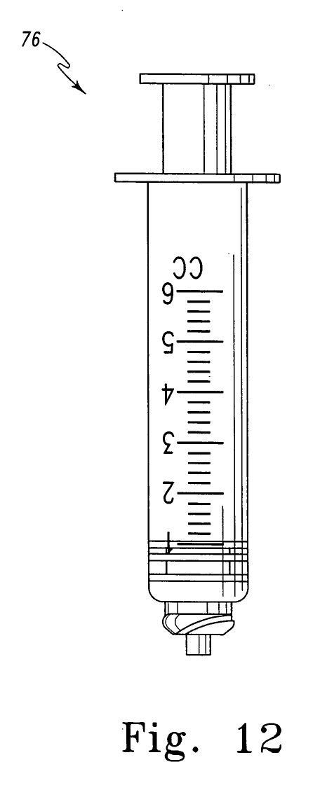

Although the above embodiments are directed to suction inserters that use bulb syringes, other hand held vacuum producing instruments may be used within the scope of the present invention. By way of example, but not of limitation, FIG. 12 shows syringe 76 that may be used to produce a vacuum. Syringe 12 is small non-electrical device such as is commercially available under the trade name MONOJECT®). Such syringes may include tips that are configured to sealingly fit with various luers.

Operation of a suction inserter in accordance with one embodiment of the invention is described with reference to FIGS. 5, 8 and 10. First, suction inserter 20 is assembled. The size of head 26 is selected such that head 26 will fit within the acetabular cup to be inserted. Head 26 is then threaded onto first end portion 44 of shaft 22 until an airtight seal is established between head 26 and shaft 22. Bulb syringe 24 is sealingly threaded onto threaded portion 54 of luer 48. The order of these steps is not critical.

In the present method, once suction inserter 20 is assembled, bulb syringe 24 is then hand gripped and compressed, expelling air out of bulb syringe 24, through internal channel 46, and out of first end portion 44. The air continues through inner channel 38 and out of head 26. Some air may also be expelled out of secondary inner channel 40. Acetabular liner 42 is then placed in the desired position on head 26 as shown in FIG. 8 while ensuring that o-rings 28 and 30 are within acetabular liner 42 and at least partly in contact with acetabular liner 42 completely around the periphery of head 26. The contact of o-rings 28 and 30 between head 26 and acetabular liner 42 creates an air tight seal shown in FIG. 8. The seal isolates gap 74 between head 14 and acetabular cup 42 from atmosphere.

At this point, the gripping compression of bulb syringe 24 is relaxed or released. Because bulb syringe 24 is elastic, it attempts to regain its original shape once the surgeon's grip is relaxed or released, thus, a vacuum is created within bulb syringe 24. The vacuum is further applied through internal channel 46 and inner channel 38 and secondary inner channel 40 to gap 74 between head 26 and acetabular cup 42. In effect, bulb syringe 24 is sucking air from all areas with which bulb syringe 24 is in fluid communication.

The vacuum thus pulls acetabular cup 42 toward head 26, further compressing o-rings 28 and 30. This is shown in FIG. 10, as acetabular liner 42 has been moved in the direction of arrow 58 in relation to its position in FIG. 8 by the vacuum resulting from movement of air in the direction of arrow 56 within internal channel 46 from inner channel 38, secondary inner channel 40, and gap 74. Thus, as shown in FIG. 10, the size of gap 74 is smaller than the size of gap 74 in FIG. 8.

At this point, acetabular cup 42 is firmly positioned upon suction inserter 20 and may be maneuvered into position in the acetabular cup. The presence of a good seal may be confirmed by monitoring the condition of bulb syringe 24. If bulb syringe 24 continues to expand after being released, then a leak is present in the system.

Those of ordinary skill in the appropriate art will appreciate that the amount of vacuum that may be created with bulb syringe 24 is related to the stiffness of bulb syringe 24, because, a stiff material attempts to assume its original shape more forcefully than a more flexible material. Thus, the stiffer the material, the greater the vacuum created. Of course, the “holding power” of the suction inserter is a combination of the frictional forces generated between the sealing surface and the acetabular cup and the vacuum between the head and the acetabular cup. Thus, as more frictional holding force is provided, less vacuum is needed to obtain the same holding power.

Once acetabular liner 42 has been positioned within the acetabular cup, bulb syringe 24 is squeezed, releasing the vacuum between head 26 and acetabular liner 42. Suction inserter 20 may then be removed.

A slightly modified procedure in accordance with the principles of the present invention is enabled by simply not fully compressing bulb syringe 24 prior to placing head 26 partially within acetabular liner 42. Even though bulb syringe 24 is not fully compressed, the release or relaxation of the compressing grip will typically provide sufficient vacuum to maintain acetabular liner 42 on head 26. Thus, when it is desired to separate acetabular liner 42 from head 26, bulb syringe may be compressed to first release the vacuum. By continuing to further compress bulb syringe 24, pressure may be applied through internal channel 46, inner channel 38 and secondary inner channel 40 into gap 74. This pressure acts against acetabular liner 42, overcoming any frictional forces and separating acetabular liner 42 from head 26.

Another modified procedure incorporating features of the present invention is enabled by the placement of one or more valves on suction inserter 20. The valve may be located at a variety of locations on bulb syringe 24 or shaft 22. When using a vent valve, acetabular liner 42 may be placed on head 26 prior to squeezing bulb syringe 24 if desired. In such a circumstance, once acetabular liner 42 is placed on head 26, the vent valve is opened and bulb syringe 24 is squeezed to force air out of bulb syringe 24. Instead of proceeding through internal channel 46, the air is expelled through the vent valve to the atmosphere. The vent valve is then shut, and bulb syringe 24 is released. A vacuum is thus applied within suction inserter 20 as described above.

Once acetabular liner 42 is positioned within an acetabular cup, the vent valve is opened to release the vacuum and suction inserter 20 is removed from the incision. Alternatively, the vent valve may be opened and then shut. This releases the vacuum and fills bulb syringe 24 with air. Bulb syringe 24 may then be squeezed to force acetabular liner 42 off of head 26. The availability of additional force for separating head 26 from acetabular liner 42 is particularly helpful when increased frictional force is present between head 26 and acetabular liner 42.

If desired, the only force used to hold an acetabular liner onto head 26 while inserting the acetabular liner may be frictional. In such a circumstance, the acetabular liner is merely forced onto head 26 and held by friction. Bulb syringe 24 may be screwed onto shaft 22 or not. Once the acetabular liner is placed in an acetabular cup, bulb syringe 24 is placed onto shaft 22 if it was not previously screwed on, and squeezed to force separation of head 26 and the acetabular liner.

Another modified method in accordance with the present invention is enabled by including an isolation valve on shaft 22, between bulb syringe 24 and head 26. In this modified method, once a vacuum is drawn and the acetabular liner is seated on head 26, the isolation valve is shut. This maintains the vacuum between head 26 and the acetabular liner, allowing bulb syringe 24 to be removed. Thus, suction inserter 20 becomes very maneuverable. Once the acetabular liner is positioned, bulb syringe 24 is replaced and the isolation valve is opened. At this point, most of the vacuum is released since bulb syringe 24 was filled with air upon its initial removal, and head 26 may be separated from the acetabular liner. Alternatively, bulb syringe 24 may be squeezed, to force the acetabular liner to separate from head 26.

Yet another method incorporating features of the present invention is discussed with reference to suction inserter 60 of FIG. 11. Initially, vent valve 70 and isolation valve 68 are shut, and stop check valve 72 is placed in the released position. Bulb syringe 66 is then squeezed, expelling air through stop check valve 72 to the atmosphere, and creating a vacuum within bulb syringe 66. After placing an acetabular liner on head 64, isolation valve 68 is opened, thus applying the vacuum within bulb syringe 66 through the internal channel of shaft 62 and the inner channels of head 64 to secure the acetabular liner to head 64. If desired, increased vacuum may be created by further squeezing of bulb syringe 66.

If removal of bulb syringe 66 is desired, such as for the purpose of increasing maneuverability, isolation valve 68 is shut. Bulb syringe 66 may then be removed without loosing the vacuum between the acetabular liner and head 64. Removal of bulb syringe 66 is eased by opening vent valve 70 to release the vacuum within bulb syringe 66.

If bulb syringe 66 has been removed, then once the acetabular liner is placed within an acetabular cup, isolation valve 68 may be opened to release the vacuum between head 64 and the acetabular liner, and suction inserter 60 may be removed. If forced separation is desired, bulb syringe 66 is reattached to shaft 62, either before or after opening isolation valve 68. Vent valve 70 is then shut and stop check valve 72 is placed in the stop position. By squeezing bulb syringe 66, air is forced through shaft 62 and the acetabular liner is forcibly separated from head 64.

If bulb syringe 66 was not previously removed, then once the acetabular liner is positioned in an acetabular cup, vent valve 70 is opened to release the vacuum between head 64 and the acetabular liner. If additional force is desired to separate the acetabular liner from head 64, then once the vacuum has been released, vent valve 70 is shut and stop check valve 72 is placed in the stop position. Squeezing bulb syringe 66 will force air through shaft 62 and the acetabular liner will be forcibly separated from head 64.

Those of ordinary skill in the relevant art will appreciate that an instrument in accordance with the present invention is very versatile and may take on a number of alternative embodiments. By way of example, but not of limitation, the head and shaft may be formed as a single unit, or the head may frictionally engage the shaft. Additionally, a single cone shaped head with a series of o-rings may be used to fit a plurality of liners of varying sizes. Moreover, there may be fewer or more inner channels and/or secondary channels in the head of an instrument. If desired, a single o-ring or other type of device may be used to create a sealed gap between an acetabular liner and the head of the instrument. Furthermore, the above described valves may be located in a number of alternative locations, and the order of many of the steps in the above described methods may be modified according to the preferences of a particular surgeon. Such alternatives and permeations are within the scope of the present invention.

1. A handheld instrument for insertion of an acetabular liner into an acetabular cup comprising:
a shaft having an internal channel therethrough and a first and a second end portion, the first end portion configured to sealingly engage with a bulb syringe;
a bulb syringe sealingly engaged with the first end portion of the shaft and operably connected to the internal channel of the shaft; and
a head having a curvilinear outer perimeter and operably attached to the second end portion of the shaft and having an inner channel therethrough operably connected to the internal channel of the shaft, the curvilinear outer perimeter of the head sized to at least partially fit within an acetabular liner.

2. The instrument of claim 1, further comprising:
a first o-ring circumscribing the curvilinear outer perimeter of the head and sized to sealingly fit between the curvilinear outer perimeter of the head and an acetabular liner.

3. The instrument of claim 2, further comprising:
a second o-ring circumscribing the curvilinear outer perimeter of the head and adjacent the first o-ring, and wherein the head further comprises:
a secondary inner channel having a first and a second end portion and operably connected at the first end portion to the internal channel and opening at the second end portion at the outer perimeter of the head between the first and second o-ring.

4. The instrument of claim 1 further comprising a valve, the valve operable to seal the internal channel such that air is not allowed to pass between the atmosphere and the internal channel through the valve.

5. The instrument of claim 1, further comprising:
a stop check valve having an inlet and an outlet, the inlet operably connected to the internal channel and the outlet operably connected to the atmosphere, such that when the stop check valve is in a non-stopped position, air from the atmosphere is not allowed to pass into the internal channel through the stop check valve but air from the internal channel is allowed to pass to the atmosphere through the stop check valve and such that when the stop check valve is in a stopped position, air from the internal channel is not allowed to pass into the atmosphere through the stop check valve; and
a valve movable between a first position and a second position and having an inlet and an outlet, the inlet operably connected to the atmosphere and the outlet operably connected to the bulb syringe, such that when the valve is in the first position, air is not allowed to pass between atmosphere and the internal channel, and when the valve is in the second position, air is allowed to pass between the atmosphere and the internal channel.

6. The instrument of claim 5, wherein the sealable valve is a stop check valve.

7. The instrument of claim 5, wherein the stop check valve is located on the bulb syringe, such that air passing between the inner channel and the atmosphere through the stop check valve passes through the bulb syringe.

8. The instrument of claim 1, wherein the shaft is bent between the first end portion and the second end portion.

9. The instrument of claim 8, wherein the shaft comprises a bend of between about 20 and about 45 degrees between the first end portion and the second end portion.

10. The instrument of claim 9, wherein the shaft comprises a bend of about 30 degrees between the first end portion and the second end portion.

11. The instrument of claim 1, wherein the head comprises an internal chamber communicating with the inner channel, and wherein the second end portion of the shaft sealingly fits within the internal chamber.

12. A method of inserting an acetabular liner into an acetabular cup comprising the steps of:
positioning an acetabular liner on the head of an instrument;
placing the acetabular liner into an acetabular cup; and
squeezing for a first time a bulb syringe connected to the instrument to expel air out of the bulb syringe.

13. The method of claim 12, wherein the step of squeezing is performed before the step of placing, the method further comprising the steps of:
applying a vacuum between the head and the acetabular liner; and
releasing the vacuum, the releasing step performed after the step of placing.

14. The method of claim 13, further comprising, after the step of applying a vacuum, the steps of:
shutting an isolation valve;
removing the bulb syringe from operable connection to the instrument;
replacing the bulb syringe into operable connection with the instrument; and
opening the isolation valve.

15. The method of claim 14, further comprising, after the step of placing the acetabular liner into an acetabular cup, the step of:
squeezing for a second time the bulb syringe to expel air out of the bulb syringe.

16. The method of claim 13, wherein the step of releasing the vacuum comprises the step of:
opening a vent valve.

17. A method of inserting an acetabular liner into an acetabular cup comprising the steps of:
squeezing the bulb syringe of an instrument a first time;
sealingly engaging the head of the instrument within an acetabular liner so as to create a sealed area between the head and the acetabular liner;
drawing a vacuum in the sealed area;
placing the acetabular liner in an acetabular cup;
removing the vacuum from the sealed area; and
separating the head from the acetabular liner in the acetabular cup.

18. The method of claim 17, wherein the step of removing the vacuum comprises the steps of:
opening a vent valve on the instrument, and wherein the step of separating the head from the acetabular liner comprises the steps of:
shutting the vent valve;
stopping a stop check valve on the instrument; and
squeezing the bulb syringe a second time to separate the head from the acetabular liner.

19. The method of claim 18, further comprising, before the step of squeezing the bulb syringe the first time, the steps of:
shutting an isolation valve on the instrument;
shutting the vent valve; and
placing the stop check valve in a non-stopped condition, and wherein the step of drawing a vacuum comprises the steps of:
opening the isolation valve; and
releasing the bulb syringe.

20. A kit providing a handheld instrument for insertion of an acetabular liner into an acetabular cup comprising:
a shaft having an internal channel therethrough and a first and a second end portion, the first end portion configured to sealingly engage with a bulb syringe, the second end configured to sealingly engage with a head; and
a plurality of heads, each head having a curvilinear outer perimeter and configured to be operably attached to the second end portion of the shaft, each of the plurality of heads having an inner channel therethrough capable of operable connection to the internal channel of the shaft, each of the plurality of heads having a curvilinear outer perimeter sized to at least partially fit within an acetabular liner.

21. The kit of claim 20, further comprising:
a bulb syringe configured to sealingly engage the first end portion of the shaft and capable of operable connection to the internal channel of the shaft.

22. The kit of claim 20, wherein each of the plurality of heads has an outer perimeter of a size different than the size of each of the other plurality of heads.

23. The kit of claim 20, wherein the plurality of heads comprises a first head, a second head and a third head, the first head having a curvilinear outer perimeter sized to at least partially fit within a 26 mm diameter acetabular liner, the second head having a curvilinear outer perimeter sized to at least partially fit within a 28 mm diameter acetabular liner, and the third head having a curvilinear outer perimeter sized to at least partially fit within a 32 mm diameter acetabular liner.

24. The kit of claim 20, wherein the plurality of heads comprises:
a first head having a curvilinear outer perimeter sized to at least partially fit within a first acetabular liner having a first diameter; and
a second head having a curvilinear outer perimeter sized to at least partially fit within a second acetabular liner having a second diameter, the first diameter different from the second diameter, and wherein the first acetabular liner and the second acetabular liner have diameters of 26 mm, 28 mm, 32 mm, 36 mm or 38 mm.

25. An instrument for insertion of an acetabular liner into an acetabular cup comprising:
a shaft having an internal channel therethrough and a first and a second end portion, the first end portion configured to sealingly engage with a hand held vacuum producing device; and
a head having a curvilinear outer perimeter and operably attached to the second end portion of the shaft and having an inner channel therethrough operably connected to the internal channel of the shaft, the curvilinear outer perimeter of the head sized to at least partially fit within an acetabular liner.

26. The instrument of claim 25, wherein the head is configured to sealingly fit within an acetabular liner.

27. The instrument of claim 25, the head comprising:
a first groove circumscribing the curvilinear outer perimeter of the head; and
a first o-ring located within the first groove and sized to sealingly fit between the curvilinear outer perimeter of the head and an acetabular liner.

28. The instrument of claim 26, the head further comprising:
a second groove circumscribing the curvilinear outer perimeter of the head and adjacent the first groove;
a second o-ring located within the first groove; and
a secondary inner channel having a first and a second end portion and operably connected at the first end portion to the internal channel and opening at the second end portion at the outer perimeter of the head between the first and second groove.

29. The instrument of claim 25 wherein the hand held vacuum producing device is a syringe.

30. The instrument of claim 28, wherein the syringe is a bulb syringe.
